(19) United States
(12) Patent Application Publication
(10) Pub. No.: US 2020/0008906 A1
(43) Pub. Date: Jan. 9, 2020

(54) REVERSIBLE THERMOPLASTIC POLYMER AND ORTHODONTIC METHOD

(71) Applicant: Mohammad IZADI, Timonium, MD (US)

(72) Inventor: Mohammad IZADI, Timonium, MD (US)

(21) Appl. No.: 16/290,662

(22) Filed: Mar. 1, 2019

Related U.S. Application Data
(60) Provisional application No. 62/637,035, filed on Mar. 1, 2018.

(51) Int. Cl. A61C 7/16; A61C 7/20; A61K 6/00

(52) U.S. Cl. CPC A61C 7/16; A61K 6/0023; A61C 7/20

(57) ABSTRACT

A reversible thermoplastic polymer for use in an orthodontic method and for eliminating a need for an acid treatment of a tooth before an orthodontic procedure, said shape-memory comprising: a material selected from the group consisting of Irgacure or Camphorquinone; Phenylpropanedione (PPD); and Lucirin TPO.

### BACKGROUND

Orthodontic devices and orthodontic procedures are continually improved toward enhancing efficacy while shortening chair time.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1A is a chart of reversibility of adhesion and images of demonstrations of the reversible adhesive property, in accordance with some embodiments.

FIG. 1B-1C are depictions of demonstrations of the reversible adhesive property of Epoxy/PCL (15.5).

FIG. 2 is a micrograph of Epoxy/PCL (15.5), in accordance with some embodiments.

FIG. 3 is a micrograph of Epoxy/PCL (15.5), in accordance with some embodiments.

FIGS. 4A-4B are drawings of a bracket design and bonded assembly, in accordance with some embodiments.

FIG. 5 is a view of a testing assembly, in accordance with some embodiments.

FIG. 6 is a drawing of use of a modulus switching adhesive, in accordance with some embodiments.

### DETAILED DESCRIPTION

The following disclosure provides many different embodiments, or examples, for implementing different features of the provided subject matter. Specific examples of components, values, operations, materials, arrangements, or the like, are described below to simplify the present disclosure. These are, of course, merely examples and are not intended to be limiting. Other components, values, operations, materials, arrangements, or the like, are contemplated. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

In earlier U.S. Pat. No. 9,895,206 for an “Adjustable Orthodontic Bracket and Method Using A Microstructured Shape Memory Polymer Surface With Reversible Dry Adhesion,” issued on Feb. 20, 2018, an adjustable orthodontic bracket includes a metal base adapted to be rigidly secured directly to a human tooth by a relatively thin layer of an orthodontic adhesive. This relatively thin layer of heat softening material fastens an orthodontic bracket directly to a tooth and subsequently heats the orthodontic material with a digital laser that allows the polymer material to reproduce a liquidous surface for adjusting the position of the bracket so that the bracket can be adjusted in all directions around the base member without overlapping the periphery of the base member.

Research has been and is being conducted to achieve new compositions of matter, new processes, and supporting characterization data for the development of new functional adhesives for use with novel orthodontic brackets and/or methods. The present project examines two distinct approaches: (i) reversible adhesion, and (ii) modulus-switching, leading to materials characterized to the extent allowing proof-of-concept assessment. In particular, bonding that has a photo initiator system (Irgacure) to help with curing the polymer initially with a curing light such as LED with a capacity of between 400-515 nm in wavelength, repositioning by heating the material, and removal of brackets to metal, glass, or donor tooth substrates are conducted and characterized with appropriate levels of quantification. Further, cytotoxicity is evaluated for biosafety/biocompatibility considerations.

Dr. Izadi (Izadi Orthodontics) has designed material and ideas for orthodontic brackets capable of being repositioned, “on the fly” by translation or rotation, in a manner that allows better application of force or moments to the associated tooth via archwire, elastic bands, or both to achieve accurate positioning of the teeth. The new bracket designs require new, functional adhesives that temporarily allow bracket manipulation by debonding (reversible adhesive approach) or softening (modulus-switch approach).

Information on reversible adhesives can be further found in:
X. Luo, R. Ou, D. E. Eberly, A. Singhal, W. Viratyaporn and P. T. Mather, “A Thermoplastic/Thermoset Blend Exhibiting Thermal Mending and and Reversible Adhesion,” ACS Applied Materials and Interfaces 1 (3) 612-620 (2009), incorporated herein by reference in its entirety; and
X. Luo, K. E. Lauber, P. T. Mather, “A Thermally Responsive, Rigid, and Reversible Adhesive,” Polymer 51 n5 1169-1175 (2010), incorporated herein by reference in its entirety.

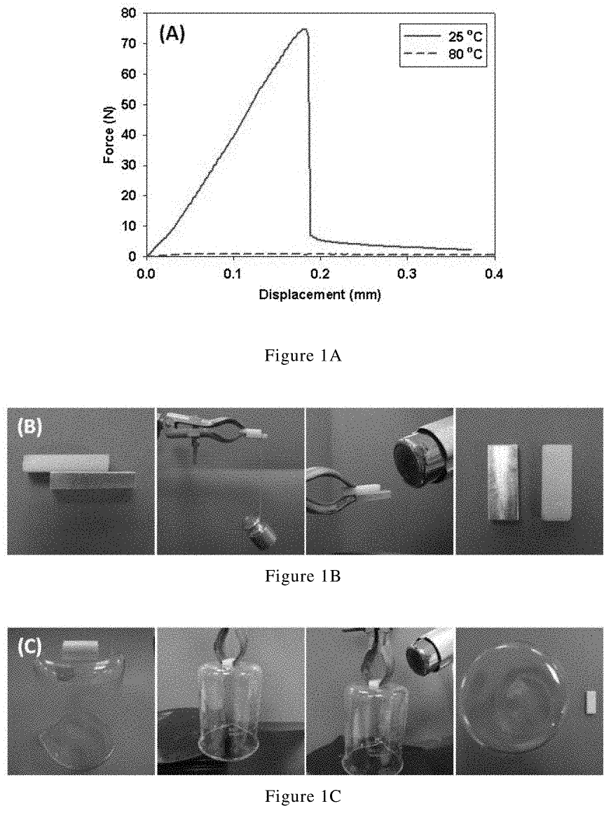

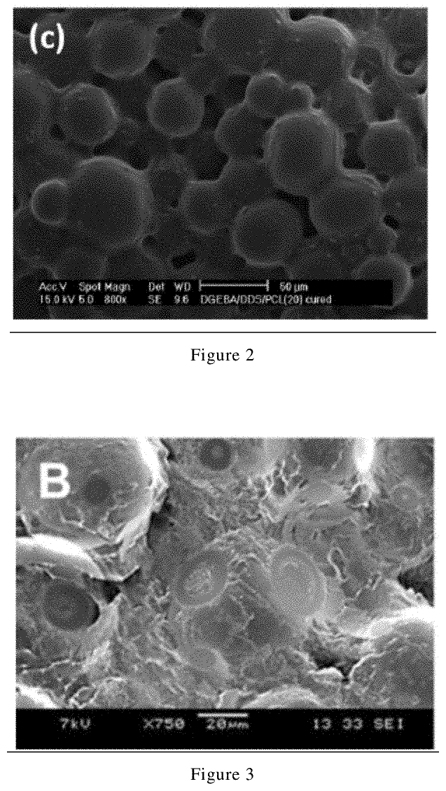

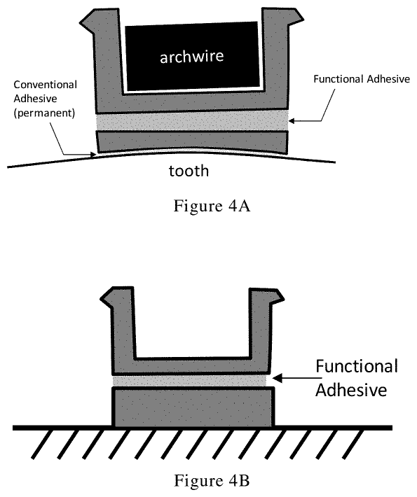

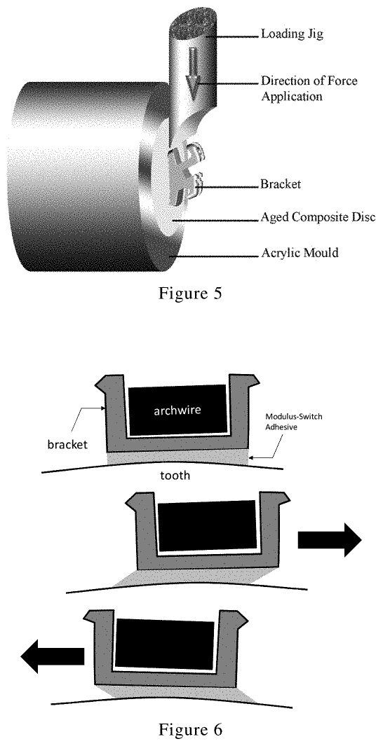

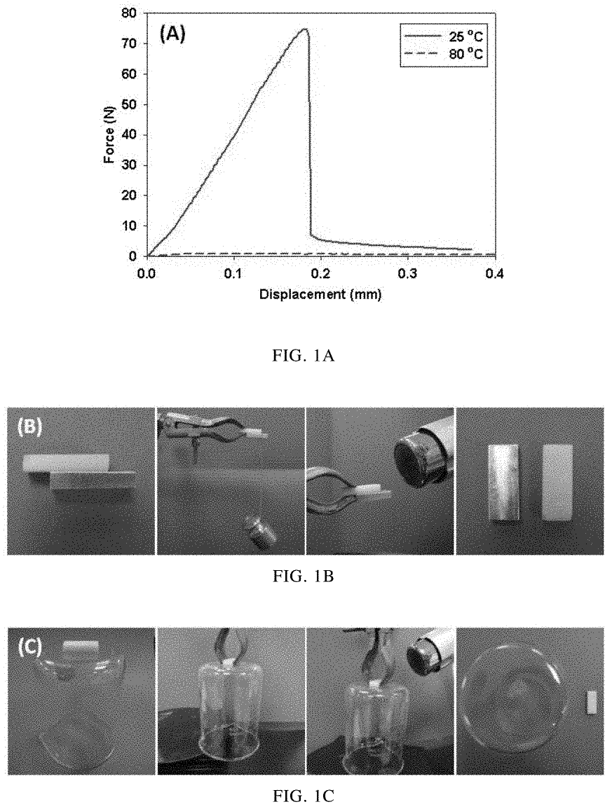

In both of these publications, polymerization-induced phase separation (PIPS) of an initially miscible thermoplastic (PCL)/thermoset (epoxy) blend, yielding a unique brick and mortar morphology exhibiting excellent reversible adhesion characteristics when bonding to glass, metal, or itself have been demonstrated. These papers clarify that the mechanism of reversible adhesion is related to the thermally reversible melting/recrystallization of the “mortar” phase (PCL), (itself, a hot-melt adhesive). Meanwhile, the “brick” phase (epoxy) maintained structural rigidity of the material, even during debonding. FIG. 1A-C from the 2010 paper, Luo, et. al, shows these interesting and potentially useful phenomena for both aluminum and glass substrates with regard to the reversibility of adhesion. FIG. 1A is a graph of tensile testing results of two aluminum bonded specimens both prepared by heating at 150° C. for 20 minutes but tested at two different temperatures of 25° C. and 80° C. FIGS. 1B and 1C are depictions of demonstrations of the reversible adhesive property of Epoxy/PCL (15.5).

For use in orthodontic brackets, this (or similar) reversible adhesive is positioned between two pieces of metal constituting the bracket, or directly between a single-part metallic bracket and the patient's tooth. During the initial procedure, the reversible adhesive is temporarily placed by the orthodontist or orthodontic assistant by heating to about 80° C. (with a light or heating tool). Refinement of the bracket configuration is executed by the orthodontist using the sequence, heat/adjust/cool, following which the bracket is firmly bonded and accurately positioned. In some embodiments, modifications to the published formulation are contemplated, as it was not developed with the orthodontic application in mind. However, it is important to note that no acid pretreatment is used in some embodiments.

Another approach to enable the function of Dr. Izadi's new bracket designs involves the use of a polymer adhesive featuring a sharp and dramatic change in elastic modulus from a glassy to a rubbery consistency, purposefully triggered by light or a heated tool executed during orthodontic adjustments. In the glassy state (normal use), the bracket is rigidly bonded to the tooth, able to transfer mechanical loads and torques from the archwire and/or elastic bands. However, in the triggered rubbery state, the bracket is able to be sheared relative to the tooth or sub-bracket metal to rotate or translate to a new position. Upon cooling (also executed with a specific tool or flushing with chilled water) the adhesive regains its glassy modulus. Errors in new placement of the bracket are easily overcome by repeating the procedure. The envisioned modulus-switching adhesive is similar to “Class I” shape memory polymers (SMP) described in:
C. Liu, H. Qin, and P. T. Mather, “Review of Progress in Shape Memory Polymers,” invited feature article J. Mater. Chem. 14, 1543-1558 (2007), incorporated herein by reference in its entirety.

Class I shape memory polymers (SMPs) are polymers featuring a glass transition temperature (T_{g}) above room temperature and covalent crosslinking between the constituent chain molecules that prevents flow above T_{g}, instead yielding rubbery, elastic behavior at those temperatures. For the present device requirements, a relatively high T_{g} that avoids premature softening when the patient exposes their mouth to hot food or drink, such as soup, coffee or tea, is used in at least some embodiments. Furthermore, above T_{g}, the elastic modulus should be very low for facile manipulation prior to cooling to fix the bracket position. On the basis of the required property tuning, the need for good adhesion to metal and tooth enamel, and biocompatibility, the families of methacrylate and acrylate copolymers can be considered, with backbone composition dictating T_{g} and crosslink density dictating elastic modulus above T_{g}.

Bracket removal following completion of orthodontic treatment is not so dramatic during debonding as to damage the native enamel of the tooth surface nor cause undue trauma or even discomfort to the patient in some embodiments. The reversible adhesive approach has the positive attribute of requiring only a mild solvent to locally remove the thermoplastic bonding residue from the tooth enamel surface.

Adequate adhesion is, in at least some embodiments, an important factor related to success of the functional adhesives in the orthodontic application. No amount of enhanced functionality would overcome a deficiency in bond strength. According to the literature, a “shear debond strength” in the range of 5.9-7.8 MPa is considered to be sufficient to withstand masticatory forces experienced in the patient's mouth. In a nutshell, a “chisel” technique is used to shear the bracket/adhesive/substrate assembly to failure and the shear bond strength taken as the peak force during debonding divided by the contact area of the bracket to the tooth through the adhesive.
“Mechanics and Mechanical Testing of Orthodontic Materials (Chapter 2)” by W. A. Brantley, T. Eliades, and A. S. Litsky, in Orthodontic Materials: Scientific and Clinical Aspects, Ed. W. A. Brantley and T. Eliades, Thiele Publishing, New York, N.Y. (2001), incorporated herein by reference in its entirety.

Reversible Adhesive

At least a portion of the present disclosure relates to testing of the PIPS-based reversible adhesive for this project. However, alternative formulation/s are usable to address deficiencies relative to adhesive requirements.

Approach

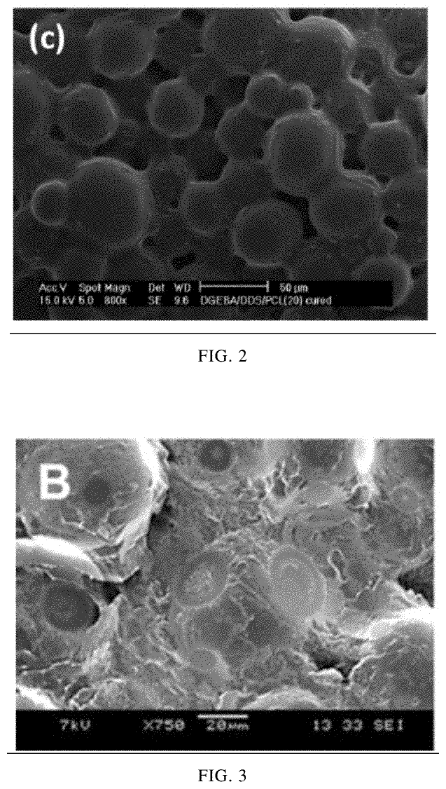

PIPS samples are prepared as described in Luo, Lauber, and Mather (2010), cited above and incorporated herein by reference in its entirety. That paper describes the processing and properties of blends of diepoxide, diamine hardener, and PCL. A thermally stable, high T_{g} epoxy blend with PCL was achieved by using a particular diamine hardener, diamino-diphenyl sulfone (DDS), and a range of PCL contents from 4 to 35 wt-% was studied. It was observed that phase inversion from epoxy matrix to PCL matrix (the desired morphology here) occurred at approximately 10%. Further, it was observed that as the PCL weight percentage was increased, the epoxy microsphere diameter decreased. Finally, it was concluded that the best properties were achieved for Epoxy/PCL (15.5) (micrographs shown in FIGS. 2 and 3), interpreted as a result of good epoxy interconnectivity.

To prepare the reversible adhesive, the refined materials preparation process of the 2010 paper listed above was used. Briefly, PCL blended with DGEBA at 20 wt-% (after dilution with DDS, 15.5 wt-% results) at 120° C. in a round-bottom, 3-necked flask with mechanical stirring and nitrogen blanket, yielding a clear mixture. Next, the temperature will be raised to 140° C. and DDS will be added at 2:1 (DEGEBA:DDS) stoichiometric equivalence, with stirring continuing for 20-30 min or until the DDS is completely melted and blended with the DGEBA/PCL blend. At this stage, the nitrogen purge is replaced with a vacuum to degas the mixture and the temperature reduced to 120° C. and degassing continued for 1 hour. The hot blend is poured into preheated silicone molds for curing at 180° C. for 3 hours, during which time PIPS occurs, yielding milky yellow specimens. Each solid adhesive will be 5 millimeters (mm)×5 mm×1 mm in size for initial characterization. The lateral dimensions will be trimmed to size using a heated knife.

Should a formulation not involving epoxy be required due to any problems with cytotoxicity testing, another system known to exhibit PIPS but an entirely different chemistry: poly(ethylene-co-vinyl-acetate) (commonly called EVA) dissolved in methyl methacrylate monomer, is usable in some embodiments. When polymerized, PIPS is known to occur (Prog. Polym. Sci. 20 119-153 (1995)) and result in a brick-mortar morphology similar to the one we have observed with the epoxy system. The formulation will require the addition of crosslinker, photoinitiator, and reactive diluents (the viscosity is expected to be high).

### Experiments and Analysis

Before testing the prepared materials as functional adhesives, we first confirm their thermal and mechanical behavior using differential scanning calorimetry (DSC) and dynamic mechanical analysis (DMA), respectively.

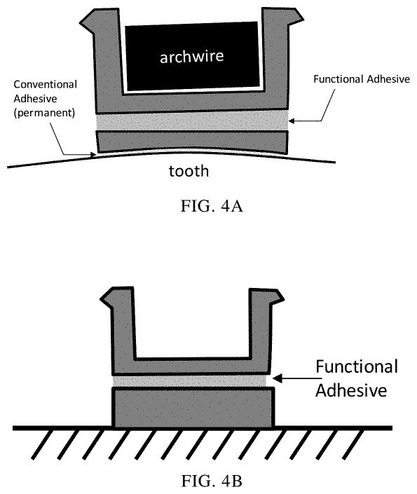

The reversible adhesives will be bonded between metallic brackets and similarly sized metallic substrates intended to mimic the two-part bracket design (as depicted in FIG. 4A) but designed to isolate the bond strength of the functional adhesive.

The bonded assembly shown in FIG. 4B should be examined qualitatively for its performance in reversible adhesion. In particular, we use hand-tools to gauge (semi-quantitatively) the bond strength at room temperature and after heating to a range of temperatures nominally exceeding the PCL melting temperature of 58° C.: 65° C., 75° C., and 85° C. Heating is conducted using a variety of methods, including a convection oven, achieving thermal equilibrium for the entire assembly, as well as a miniature heating gun, and a contact heater of the soldering iron variety. From these qualitative experiments, we gained a quick understanding of the bonding, debonding, and re-bonding functionality. Adjustments are made to the reversible adhesive geometry (especially thickness, but also lateral dimensions), composition, and heating method until iterations reveal a methodology that satisfies the application requirements. The level of adhesive remnants on the tooth surface upon bracket removal can be investigated through scanning electron microscopy (SEM); SEM micrographs are taken of teeth pre-adhesion, post-adhesion, and post-solvent cleaning to determine the total adhesive left on the tooth upon bracket removal and the ability to clean the tooth without requiring abrasion. At that point, we have to proceed to shear bond strength testing.

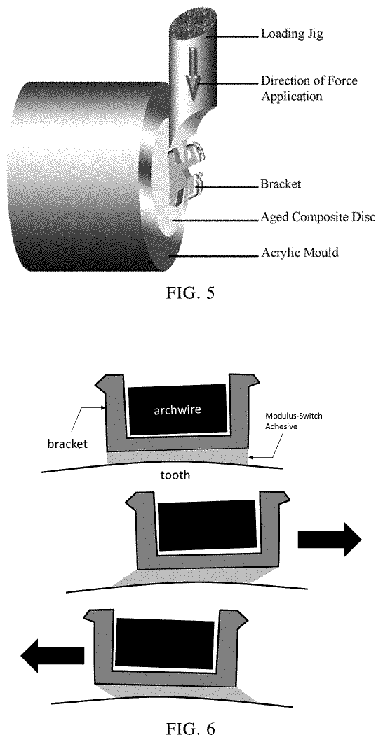

Shear bond strength (SBS) can be examined using custom-built grips in conjunction with a tensile testing apparatus. Uniquely, this instrument is outfitted with a “bio-bath” immersion chamber (http://www.testresources.net/accessories/biomedical-baths) for testing materials under physiological conditions, usually PBS buffer and T=37° C. Following the methods described in the book chapter by W. A. Brantley, T. Eliades, and A. S. Litsky (above), the assemblies will be tested using the geometry shown in FIG. 5. We target a shear bond strength exceeding 5 MPa (500 N/cm²) at physiological conditions and 100× less than that when activated by heat. The effect of thermal cycling and bracket repositioning on the SBS can be investigated by performing 3 heat/adjust/cool cycles and measuring the SBS following each cycle. To examine shear-based repositioning of the brackets, a distinct loading jig capable of maintaining the bracket in contact with the substrate (tooth) during shearing-type displacement is used, in some embodiments. The same arrangement shown in FIG. 5 for SBS testing is used to characterize the second approach to functional adhesives, discussed below: Modulus-Switching Adhesive.

Modulus Switching Adhesive

Here, we seek to utilize a lightly crosslinked, glassy polymer featuring a well-defined glass-transition temperature and a large drop in elastic modulus at that point. FIG. 6 is a schematic drawing of use of a modulus switching adhesive, in accordance with some embodiments. In this approach, the bracket is bonded to the tooth directly by the functional adhesive. To adjust the bracket position, the functional adhesive is heated to a softened state and (in at least some embodiments, with the same tool) displaced to the desired position. Cooling with chilled water allows “fixing” in the new position. Beyond 100% shear strain (displacement/thickness×100%), the displacement force will grow to be too large for convenient manipulation. As such, the thickness should be larger than a conventional adhesive and correspond roughly to the desired lateral displacement of approximately 1-2 mm.

Approach

Building upon examining Class-I shape memory polymers, we formulate a modulus switching adhesive with the desired characteristics. We utilize a functionalized methacrylate monomer with the generic structure CH₂═C(CH₃)—R, where R is selected to yield the proper T_{g} in fully cured form. For finer tuning of the T_{g}, copolymerization using two methacrylate monomers is able to be used, with a T_{g} ranging from 20-115° C. achievable. This monomer/comonomer is crosslinked with a diacrylate (or diacrylamide) with general structure CH₂=CH—R′—CH═CH₂ and with a concentration small enough to yield a large modulus switch upon heating, but large enough to provide a mechanically robust network structure indicated by a gel-fraction (mass-remaining after extraction in a good solvent) of at least 90%. Finally, the formulation is rendered photo-curable by addition of a photoinitiator (Irgacure) tuned to the curing light wavelength commonly used in orthodontics. Viscosity is increased for handling purposes by incorporation of 1-10% of poly(methyl methacrylate) linear polymer.

### Experiments and Analysis

Monomers are able to be mixed in standard laboratory vials until all components are well dissolved and a homogeneous, transparent solution results. Two types of specimens are prepared: films for basic polymer characterization and adhesively bonded brackets. The former specimens are prepared in glass-slide molds for curing in a possibly custom-build UV cure chamber. For this, liquid precursor is pipetted into the mold and the mold exposed to UV light (top and bottom illumination) for a range of times. The effect of cure time and adhesive formulation are examined using DSC and DMA as described above, as well as gel fraction. For the case of adhesively bonded brackets, solutions are pipetted onto a glass substrate and the bracket applied for bonding. Excess viscous fluid is removed with a knife edge, in at least some embodiments. The adhesive is cured by light exposure through the glass substrate for variable times, selecting for detailed characterization the cure time leading to maximum apparent SBS.

Functional bracket testing is semi-quantitative in nature, examining the degree to which the bracket can be displaced laterally before adhesive or cohesive failure. Stability of the fixed state is examined by exposure to water of increasing temperature for 10 seconds over the range of temperatures from 0-100° C. While it is not expected, if functional testing reveals the modulus switching adhesive is too stiff (in the rubbery state) for sufficient displacement, an alternate approach is usable wherein the adhesive is configured in a sparse, waffled pattern. This decreases the apparent modulus, while preserving SBS.

Following bracket removal, the adhesive removal index is determined using SEM, as discussed for the reversible adhesive above. Since the modulus switching adhesive will be covalently crosslinked, removal using a solvent alone is not feasible. Thus, to remove remaining adhesive, tungsten carbide burs will be used. SEM micrographs of teeth post-cleaning with the tungsten carbide burrs reveal the effectiveness of adhesive removal and any damage to the enamel.

The Initiator System

Incoming photons are absorbed by a photo-initiator which, when activated enables the formation of free radicals and thus triggers the polymerization reaction. Knowledge of the absorption spectrum of a material's photo-initiator chemistry is critical for effective polymerization. The guiding principle that dictates the efficiency of a photo polymerization reaction is how much light energy is absorbed by the photo-initiator in the system, which is dependent on the efficiency of the Light Curing Unit (LCU) and the total energy concept. This means that, while light intensity is important, the more important factor is how much of the emitted light effectively matches the absorption spectrum of the photo-initiator. Only when the wavelength of the LCU matches the maximum absorption of a material's photo-initiator does efficient light polymerization occur.

There are three initiators currently in general use:
Camphorquinone;
Phenylpropanedione (PPD); and
Lucirin TPO

These, like all photo-initiators, are able to absorb only the photons of a specific spectral range. Camphorquinone is the most commonly used initiator molecule. The peak sensitivity of camphorquinone is near 470 nanometers (nm) in the blue wavelength range. As camphorquinone has an intense yellow color due to its absorption properties, initiators such as phenylpropanedione (PPD) and Lucirin TPO are often substituted in part or in whole. The latter two initiators have absorption profiles closer to the ultraviolet (UV) region of the spectrum. Most Halogen LCUs have a spectral range around 425-515 nm which allows possible activation of all of the above photo-initiators. Conventional Light Emitting Diode (LED) LCUs had a spectral wavelength of 430-490 nm, but more recent LED LCUs have a range between 400-515 nm, more closely matching the absorption spectrum of camphorquinone (CQ). A recent LED LCU (Bluephase®) has a range of 380-515 nm which can activate Lucerin and PPD, though an adequate Depth of Cure of Resin Based Composites based on noncamphorquinone photo-initiators has yet to be established for contemporary materials and light sources.

It is noteworthy to mention that in a classic bonding scenario utilizing composite resins, we need to acid etch the enamel surface with a %37 Phosphoric acid for about 45 to 60 seconds to roughen the enamel surface for a better adhesion. Due to the bonding capability of this thermoplastic bonding material, it is very promising in eliminating the need for acid etching the enamel surface; which potentially can prevent the teeth from becoming hypo-calcified during this procedure.

The use of a light cure makes the procedure very simple and conventional in what orthodontists are used to, and of course utilizing a laser point to generate localized heat within a safe range in softening the material in seconds, not only allows maneuvering the bracket easily but also can be harmless to the tooth and its pulp structure.

Cytotoxicity Testing

Those compositions most favorably meeting the above requirements will be subjected to cytotoxicity testing using the MTT assay and producing statistically meaningful cell viability percentages. In this test, cells are cultured in contact with media that is in direct contact with the materials being studied, but not in contact with the material itself. This is the preferred approach to avoid the possibility of a false negative result that might stem simply from cell adhesion (or not) on the adhesives.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A reversible thermoplastic polymer for use in an orthodontic method and for eliminating any need for an acid treatment of a tooth before an orthodontic procedure, said polymer comprising a shape-memory material.

2. The polymer of claim 1, wherein the shape-memory material comprises:
a material selected from the group consisting of Irgacure or
Camphorquinone;
Phenylpropanedione (PPD); and
Lucirin TPO.

3. The polymer of claim 1, wherein the shape-memory material comprises a reversible adhesive or a modulus switching adhesive.

4. The polymer of claim 3, wherein the reversible adhesive comprises a thermally stable, high T_{g} epoxy blend with PCL.

5. The polymer of claim 4, wherein the reversible adhesive is Epoxy/PCL (15.5).

6. The polymer of claim 4, wherein the reversible adhesive comprises a poly(ethylene-co-vinyl-acetate) dissolved in a methyl methacrylate monomer.

7. The polymer of claim 3, wherein the modulus switching adhesive comprises a functionalized methacrylate monomer with the generic structure CH₂═C(CH₃)—R, where R is selected to yield the proper T_{g} in fully cured form.

8. An orthodontic method for attaching a base of an orthodontic bracket directly to a human tooth with conventional acid etching of the enamel but to emphasize on achieving a desirable bond strength without any acid-etching pretreatment.

9. A method for affixing a dental bracket directly to a human tooth for an initial period of time and for repositioning and re-bonding the dental bracket on said tooth comprising:
providing a dental bracket and an archwire that fits within said bracket for applying a force against said bracket to thereby straighten said tooth; and
applying a thin layer of a reversible bonding material on said tooth and automatically activating said bonding material; and
returning said bonding material to its glass-like form.
